(19) United States
(12) Patent Application Publication
(10) Pub. No.: US 2006/0052350 A1
(43) Pub. Date: Mar. 9, 2006

(54) Crystalline forms of 1,24(S)-dihydroxy vitamin D2

(76) Inventors: Alexei Ploutno, Bat-Yam (IL); Anchel Schwartz, Rehovot (IL); Koby Wolfman, Petah Tikva (IL); Judith Aronhime, Rehovot (IL)

Correspondence Address: KENYON & KENYON, ONE BROADWAY, NEW YORK, NY, 10004, US

(21) Appl. No.: 11/173,514

(22) Filed: Jul. 1, 2005

Related U.S. Application Data
(60) Provisional application No. 60/584,844, filed on Jul. 1, 2004.
(60) Provisional application No. 60/612,914, filed on Sep. 23, 2004.

(51) Int. Cl. A61K 31/59; C07C 401/00

(52) U.S. Cl. 514/167; 552/653

(57) ABSTRACT

Provided are novel crystalline forms of 1,24-(S)-dihydroxy vitamin D2, including hydrates and solvates, and methods for making them. Also provided are pharmaceutical and nutraceutical compositions containing the novel crystalline forms.

### RELATED APPLICATIONS

This application claims the benefit of U.S. provisional Application No. 60/584,844, filed Jul. 1, 2004; and U.S. provisional Application No. 60/612,914, filed Sep. 23, 2004; the contents of all which are incorporated herein.

### FIELD OF INVENTION

The present invention relates to the solid state properties of 1,24(S)-dihydroxy vitamin D₂.

### BACKGROUND OF THE INVENTION

Vitamin D is a fat-soluble vitamin. It is found in food, but also can be made in the body after exposure to ultraviolet rays. Vitamin D is known to exist in several chemical forms, each with a different activity. Some forms are relatively inactive in the body, and have limited ability to function as a vitamin. The liver and kidney help convert vitamin D to its active hormone form. The major biologic function of vitamin D is to maintain normal blood levels of calcium and phosphorus. Vitamin D aids in the absorption of calcium, helping to form and maintain healthy bones. The structure of 1α,24(S)-dihydroxy vitamin D₂ is shown below:

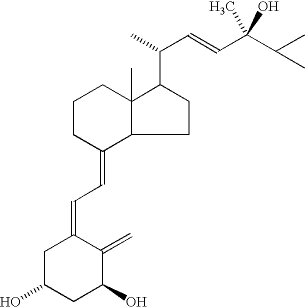

The present invention relates to the solid state structural and physical properties of 1,24(S)-dihydroxy vitamin D₂. The solid state structures can be influenced by controlling the conditions under which 1,24(S)-dihydroxy vitamin D₂ is obtained in solid form. Solid state physical properties influenced by solid state structures include, for example, the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Another important solid state property of a pharmaceutical compound that can be influenced by its solid state structure is its rate of dissolution in aqueous fluid. The rate of dissolution of an active ingredient in a patient's stomach fluid can have therapeutic consequences since it imposes an upper limit on the rate at which an orally-administered active ingredient can reach the patient's bloodstream. The rate of dissolution is also a consideration in formulating syrups, elixirs and other liquid medicaments. The solid state form of a compound is also reported to affect its behavior on compaction and its storage stability.

These practical physical characteristics are influenced by the conformation and orientation of molecules in the unit cell, which defines a particular crystalline (polymorphic) form of a substance. The crystalline form may give rise to thermal behavior different from that of the amorphous material or another crystalline form.

Thermal behavior of a compound, that is changes in state or physical characteristics, can be measured in the laboratory by such techniques as capillary melting point, thermogravimetric analysis (TGA), and differential scanning calorimetry (DSC). Thermal behavior has been applied to distinguishing some crystalline forms of a compound from others. A particular crystalline form can and typically does give rise to distinct spectroscopic properties that may be detectable by powder X-ray crystallography, solid state 1³C NMR spectroscopy, and infrared spectroscopy.

There is a need for crystalline forms of 1,24(S)-dihydroxy vitamin D₂ and for methods of making the crystalline forms of 1,24(S)-dihydroxy vitamin D₂.

### SUMMARY OF THE INVENTION

In one aspect, the present invention relates to hydrates of 1,24(S)-dihydroxy vitamin D₂ hydrate. The hydrates of the present invention preferably contain between about 1% to about 4% water. Preferably, the hydrate provided in the present invention is a monohydrate, a hemihydrate or a sesquihydrate.

In yet another aspect, the present invention relates to solvates of 1,24(S)-dihydroxy vitamin D₂. Prefered solvates of the present invention are acetonates (i.e. acetone solvates).

In one aspect, the present invention relates to a crystalline form of 1,24(S)-dihydroxy vitamin D₂ (denominated Form A), characterized by X-ray reflections at about 14.2, 16.2, 16.6, 18.4, and 22.1°±0.2° 2θ.

In another aspect, the present invention relates to a crystalline form of 1,24(S)-dihydroxy vitamin D2 (denominated Form B), characterized by X-ray reflections at about 13.6, 15.3, 16.2, 17.1, and 17.6°±0.2° 2θ.

In yet another aspect, the present invention relates to a crystalline form of 1,24(S)-dihydroxy vitamin D2 (denominated Form C), characterized by X-ray reflections at about 14.7, 15.6, 16.2, and 17.1°±0.2° 2θ.

In a further aspect, the present invention relates to a crystalline form of 1,24(S)-dihydroxy vitamin D2 (denominated Form D), characterized by X-ray reflections at about 13.4, 14.5, 15.0, and 16.8°±0.2° 2θ.

In another aspect, the present invention relates to a method of making crystalline 1,24(S)-dihydroxy vitamin D₂ Form A, including the steps of: providing a solution of 1,24(S) dihydroxy vitamin D₂ in acetone, cooling the solution to a temperature of about 0° to about −20° C., and maintaining the reaction mixture for at least about 15 hours, whereby a precipitate of the crystalline form is obtained, and, recovering the crystalline Form A.

Preferably, the solution is cooled to a temperature of about −18° C. Alternatively, the solution is initially cooled to a temperature of about 0°, followed by a further cooling to a temperature of about −18° C.

Preferably, prior to cooling, the solution is concentrated to from about 70% to about 85% of its initial volume.

In another aspect, the present invention relates to a method of making crystalline 1,24(S)-dihydroxy vitamin D₂ Form B and, including the steps of: providing a solution of 1,24(S)-dihydroxy vitamin D₂ in a mixture of methyl formate and water, about 50:1 on a volume basis, cooling the provided solution to a temperature of about 0° C. to about −20° C., maintaining the reaction mixture for a period of about 16 to about 20 hours, whereby a precipitate of the crystalline form is obtained, and recovering the crystalline Form B.

Preferably, the solution is initially cooled to a temperature of about 0°, and maintained for about an hour, followed by a further cooling to a temperature of about −18° C.

Form B may also be obtained by the method including the steps of: providing a solution of 1,24(S) dihydroxy vitamin D₂ in acetone, combining the solution with water, cooling the solution to a temperature of about 0°, maintaining the solution at about 0° C. for at least about 1.5 hours, whereby a precipitate of the crystalline form is obtained, and, optionally, recovering the crystalline form B.

In yet another aspect, the present invention relates to a method of making acrystalline form of 1,24(S)-dihydroxy vitamin D₂ denominated Form C, including the steps of: providing a solution of 1,24(S)-dihydroxy vitamin D₂ in ethyl acetate, cooling the solution to a temperature of about −10° C. to about −20° C., maintaining the solution for about 5 to about 20 hours, whereby a precipitate of the crystalline form is obtained, and ecovering the crystalline Form C.

Preferably, the solution is cooled to a temperature of about −18° C.

Preferably, prior to cooling, the solution is concentrated to from about 60% to about 80% of its initial volume, especially at reduced pressure.

In yet a further aspect, the present invention relates to the method of making crystalline 1,24(S)-dihydroxy vitamin D₂ Form D, including the steps of: providing a solution of 1,24(S)-dihydroxy vitamin D₂ in ethyl acetate, cooling the provided solution to a temperature of about 0° C. over a time period of about 1 hour, further cooling the reaction mixture to a temperature of about −10° to about −20° C., maintaining the reaction mixture for about 16 to about 19 hours, whereby a precipitate of the crystalline form is obtained, and recovering the crystalline Form D.

Preferably, prior to cooling, the solution is concentrated to about 60% to about 80% of its initial volume.

In still yet a further aspect, the present invention relates to a pharmaceutical or nutraceutical compositions, optionally formulated into a dosage form, especially a solid oral dosage form, that includes at least one pharmaceutically acceptable excipient and one or more of the crystalline forms of 1,24(S)-dihydroxy vitamin D₂ denominated herein as form A, form B, form C, and form D.

### BRIEF DESCRIPTION OF THE FIGURES

FIG. 1 illustrates the X-ray powder diffraction diagram for 1,24(S)-dihydroxy vitamin D₂ Form A.

FIG. 2 illustrates the X-ray powder diffraction diagram for 1,24(S)-dihydroxy vitamin D₂ Form B.

FIG. 3 illustrates the X-ray powder diffraction diagram for 1,24(S)-dihydroxy vitamin D₂ Form C.

FIG. 4 illustrates the X-ray powder diffraction diagram for 1,24(S)-dihydroxy vitamin D₂ Form D.

### DETAILED DESCRIPTION OF THE INVENTION

The present invention provides crystalline forms of 1,24(S)-dihydroxy vitamin D₂, and methods for making them. The present invention further provides pharmaceutical or nutraceutical compositions containing the herein disclosed crystalline forms of 1,24(S)-dihydroxy vitamin D₂.

In one embodiment, the present invention provides 1,24(S)-dihydroxy vitamin D₂ hydrate. The hydrate of the present invention preferably contains between about 1% to about 4% water. Preferably the hydrate of 1,24(S)-dihydroxy vitamin D₂ is selected from the group consisting of: a hemihydrate, a monohydrate and a sesquihydrate.

In another embodiment, the present invention provides crystalline solvates of 1,24(S)-dihydroxy vitamin D₂. Preferably the solvate of 1,24(S)-dihydroxy vitamin D₂ is acetonate. Preferably, the acetone content in the acetonate conforms with the content of a hemi-acetonate.

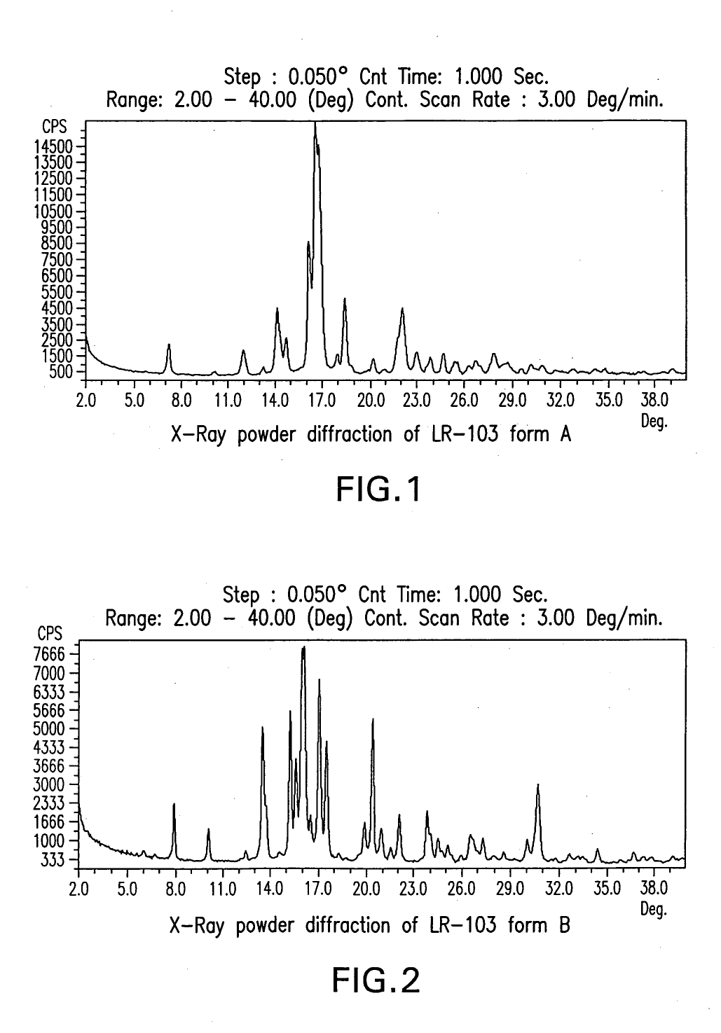

In one embodiment, the present invention provides a crystalline form of 1,24(S)-dihydroxy vitamin D₂, herein denominated form A, characterized by X-ray reflections at about 14.2, 16.2, 16.6, 18.4, and 22.1 degrees two-theta±0.2 degrees two-theta. Form A may be further characterized by X-ray reflections at about 7.2, 12.0, 14.8, 23.0, 23.8, 24.7, and 27.9 degrees two-theta±0.2 degrees two-theta. A representative powder X-ray diffraction diagram for form A is given in FIG. 1. Form A can exist as a hemiacetonate of 1,24(S)-dihydroxy vitamin D₂.

In another embodiment, the present invention provides a crystalline form of 1,24(S)-dihydroxy vitamin D₂, herein denominated form B, characterized by X-ray reflections at 13.6, 15.3, 16.2, 17.1, and 17.6 degrees two-theta±0.2 degrees two-theta. Form B may be characterized further by X-ray reflections at 8.0, 10.1, 15.6, 20.4, 22.1, 23.9, and 30.8 degrees two-theta±0.2 degrees two-theta. A characteristic powder X-ray diffraction diagram of form B is given in FIG. 2. Form B can exist as a hydrate of 1,24(S)-dihydroxy vitamin D₂.

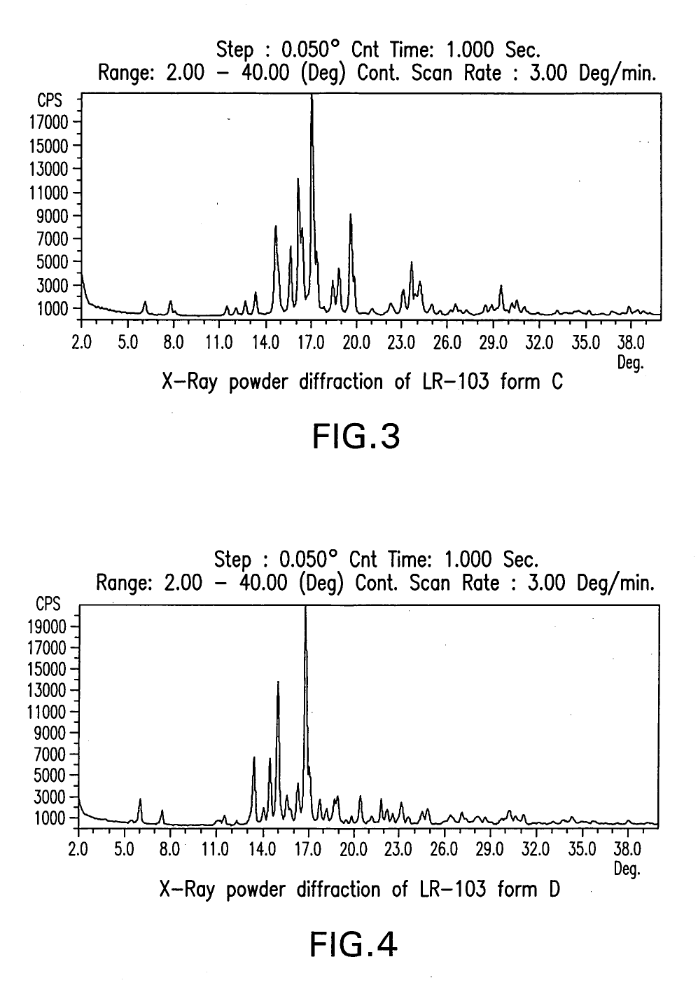

In a further embodiment, the present invention provides a crystalline form of 1,24(S)-dihydroxy vitamin D₂, herein denominated form C, characterized by X-ray reflections at 14.7, 15.6, 16.2, and 17.1 degrees two-theta±0.2 degrees two-theta. Form C is characterized further by X-ray reflections at 6.2, 13.4, 18.4, and 18.8 degrees two-theta±0.2 degrees two-theta. A representative powder X-ray diffraction diagram of form C is given in figure FIG. 3. Form C can exist as a hydrate of 1,24(S)-dihydroxy vitamin D₂ in the hemihydrate state.

Forms B and C of the present invention maintain their crystal structure, that is, they do not transform to another crystalline form, when exposed to 100% relative humidity for about 1 week at room temperature.

In yet another embodiment, the present invention provides a crystalline form of 1,24(S)-dihydroxy vitamin D₂, herein denominated form D, characterized by X-ray reflections at 13.4, 14.5, 15.0, and 16.8 degrees two-theta±0.2 degrees two-theta. Form D is characterized further by X-ray reflections at 6.0, 15.6, 16.4, 17.8, 20.5, 21.8, 23.1, 24.6, and 24.9 degrees two-theta±0.2 degrees two-theta. A representative powder X-ray diffraction diagram of form D is given in FIG. 4. Form D can exist as a sesquihydrate of 1,24(S)-dihydroxy vitamin D₂ in the sesquihydrate state.

In further embodiments, the present invention provides methods for making crystalline forms of 1,24(S)-dihydroxy vitamin D₂ that include the general steps of providing a solution of 1,24(S)-dihydroxy vitamin D₂ in a solvent or mixture of solvents selected according to the crystalline form desired; cooling, continuously or stepwise, the solution to a temperature between about 0° C. and about −20° C., and optionally, maintaining the cooled solution at the ultimate or, in the case of stepwise cooling an intermediate, temperature, or at both temperatures, for a holding time. Preferably, the provided solution is agitated (e.g. stirred) during the cooling and any holding step(s).

The solution can be provided by any convenient means, for example by dissolving the 1,24(S)-dihydroxy vitamin D2 in the desired solvent or mixture of solvents. The provided solution can be the product obtained directly from an earlier-in-time unit operation.

When the solution is provided by dissolving 1,24(S)-dihydroxy vitamin D₂ in a solvent, the 1,24(S)-dihydroxy vitamin D₂ dissolved can be any crystalline or amorphous form of 1,24(S)-dihydroxy vitamin D₂, including any solvates or hydrates. The form of the 1,24(S)-dihydroxy vitamin D₂ for the dissolving step, when used, is not important. As above, the solvent for the dissolving step is chosen according to the crystalline form desired. Useful solvents include acetone, water, methyl formate, ethyl acetate, and combinations thereof.

The amount of solvent in the provided solution is sufficient to dissolve the 1,24(S)-dihydroxy vitamin D₂ and maintain it in solution at about room temperature. If desired, the solution can be filtered, through glass wool for example, prior to the precipitation step to remove undissolved particles.

In this and other embodiments of the present invention, the starting material used for the processes of the present invention may be synthesized according to the methods known in the art, such as the one provided in U.S. Pat. No. 5,786,348.

The starting material used for the processes of the present invention may be any crystalline or amorphous form of 1,24(S)-dihydroxy vitamin D₂, including any solvates and hydrates. With processes where 1,24(S)-dihydroxy vitamin D₂ goes into solution, the form of the starting material is of minimal relevance since any solid state structure is lost in solution. With suspension and drying processes, the starting material may sometimes make a difference, as one of skill in the art would appreciate.

In particular embodiments, the provided solution is concentrated prior to cooling. When the provided solution is concentrated, concentration is conveniently accomplished at reduced pressure, less than 100 mm Hg, at about 30° C. The solution is preferably concentrated to about 60% to about 85% of its initial volume. Typically, the concentration of the provided solution is between about 1% and about 5% on a weight-per-volume basis. One of ordinary skill in the art can easily determine the sufficient amount of solvent.

At the end of the cooling step (and maintaining time, if any), the desired crystal form is recovered by conventional means. The precipitate can be recovered by any means known in the art including, but not limited to, filtration, centrifugation, and decanting. Preferably, the precipitate is recovered by filtration. The precipitate may be recovered from any composition containing the precipitate and the solvent including, but not limited to, a suspension, solution, slurry, or emulsion.

The process of particular embodiments can further include washing the precipitate.

The processes of particular embodiments can further include drying the recovered precipitate. In those embodiments in which drying is used, drying takes place at a temperature of about 28° C. for about 6 hours to overnight. In particular embodiments, drying takes place in a vacuum oven at high vacuum, for example under less than about 5 mm Hg, for about 6 to about 8 hours.

Thus, in one embodiment, the invention provides a process for making crystalline 1,24(S)-dihydroxy vitamin D₂ form A including the steps of crystallizing form A from a solution of 1,24(S)-dihydroxy vitamin D₂ in acetone; and recovering the precipitate. The solution may be filtered, through glass wool for example, prior to the precipitation step to remove undissolved particles. The solution is preferably concentrated before the crystallization step, for example under reduced pressure at a temperature of about 30° C., to about 70% to about 85% of its initial volume. Preferably, the solution is agitated during precipitation. The precipitation step includes cooling the solution. The cooling can be performed continuously or in a stepwise manner. In a preferred embodiment, cooling is continuous to a temperature of about 0° C. to about −20° C., followed by maintaining the resulting mixture for about 16 hours. Preferably, the cooling is performed to a temperature of about −18° C.

Alternatively, cooling is conducted in a stepwise manner by first cooling the solution to about 0° C. over about 1 hour and then cooling to a temperature of about −10° C. to about −20° C., preferably to a temperature of about −18° C. and maintaining for about 4 hours. The resulting precipitate is recovered, preferably by filtration. The process can include the optional steps of washing and drying the precipitate.

In a further embodiment, the invention encompasses processes for making crystalline 1,24(S)-dihydroxy vitamin D₂ form B including the step of crystallizing the crystalline form from a solution of 1,24(S)-dihydroxy vitamin D₂ in a combination of water and either acetone (about 1:3 on a volume basis) or methyl formate (about 1:50 on a volume basis). The solvents can be combined simultaneously or sequentially.

In a particular embodiment the 1,24(S)-dihydroxy vitamin D₂ is dissolved in a combination of about 2% water in methyl formate. The solution is optionally filtered prior to the precipitation step to remove undissolved particles. Preferably, the solution is agitated during the precipitation (crystallization). The crystallization step preferably includes cooling the solution.

In a particular embodiment for making form B of the present invention, crystallization is performed continuously by cooling to a temperature of about 0° C. to about −20° C. for about 16 to about 20 hours. Preferably, crystallization is performed stepwise by first cooling the solution to a temperature of about 0° C. over a time period of about 1 hour, and then cooling to a temperature of about −18° C. and maintaining the reaction mixture at this temperature for about 16 to about 19 hours. The process can optionally include washing and drying the precipitate.

In another embodiment, the 1,24(S)-dihydroxy vitamin D₂ is first dissolved in acetone and then the solution is combined with water (about 3:1 on a volume basis). Preferably, the solution is agitated during the precipitation (crystallization). The crystallization step preferably includes cooling the solution.

In a particular embodiment, precipitation is performed by cooling the solution to a temperature of about 0° C. over a time period of about 1.5 hours. The process can optionally include washing and drying the precipitate.

In another embodiment, the invention provides a process for making crystalline 1,24(S)-dihydroxy vitamin D₂ form C including the steps of providing, a solution of 1,24(S)-dihydroxy vitamin D₂ in ethyl acetate, cooling the solution directly to a temperature of −10° to −20° C., preferably to a temperature of about −18° C., whereby a precipitate forms. The solution is preferably agitated during the cooling step. The solution is preferably maintained at a temperature of −10° to −20° C. for about to about 20 hours. More preferably, the solution is maintained for about 18 hours.

Prior to cooling, the solution is optionally filtered. Preferably, the solution, filtered or not, is concentrated prior to the cooling step to about 60% to about 80%, preferably about 70%, of its initial volume.

In still yet another embodiment, the present invention provides a process for making crystalline 1,24(S)-dihydroxy vitamin D₂ form D including the steps of providing a solution of 1,24(S)-dihydroxy vitamin D₂ in either methyl formate or ethyl acetate, cooling the solution to about 0° C. for a period of about 1 hour, then cooling the resulting mixture to a temperature of about −10° C. to about −20° C., preferably about −18° C., and maintaining the reaction mixture at this temperature for about 16 to about 24 hours.

Prior to cooling, the solution is preferably concentrated to about 60% to about 80% of its initial volume.

In still yet a further embodiment, the present invention provides pharmaceutical or nutraceutical compositions containing one ore more of the crystalline forms of 1,24(S)-dihydroxy vitamin D₂ of the present invention denominated forms A, B, C, and D.

Pharmaceutical or nutraceutical formulations of the present invention contain crystalline 1,24(S)-dihydroxy vitamin D₂ such as one of those disclosed herein, or 1,24(S)-dihydroxy vitamin D₂ purely amorphous, optionally in mixture with other form(s) of 1,24(S)-dihydroxy vitamin D₂. 1,24(S)-dihydroxy vitamin D₂ that is crystallized by the processes of the present invention is ideal for pharmaceutical formulation. In addition to the active ingredient(s), the pharmaceutical compositions of the present invention may contain one or more excipients. Excipients are added to the composition for a variety of purposes.

Diluents increase the bulk of a solid pharmaceutical composition, and may make a pharmaceutical dosage form containing the composition easier for the patient and caregiver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Glidants can be added to improve the flowability of a non-compacted solid composition and to improve the accuracy of dosing. Excipients that may function as glidants include colloidal silicon dioxide, magnesium trisilicate, powdered cellulose, starch, talc and tribasic calcium phosphate.

When a dosage form such as a tablet is made by the compaction of a powdered composition, the composition is subjected to pressure from a punch and die. Some excipients and active ingredients have a tendency to adhere to the surfaces of the punch and die, which can cause the product to have pitting and other surface irregularities. A lubricant can be added to the composition to reduce adhesion and ease the release of the product from the die. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol and tartaric acid.

Solid and liquid compositions may also be dyed using any pharmaceutically acceptable colorant to improve their appearance and/or facilitate patient identification of the product and unit dosage level.

In liquid pharmaceutical compositions of the present invention, 1,24(S)-dihydroxy vitamin D₂ and any other solid excipients are dissolved or suspended in a liquid carrier such as water, vegetable oil, alcohol, polyethylene glycol, propylene glycol or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the present invention may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar may be added to improve the taste.

Preservatives and chelating agents such as alcohol, sodium benzoate, butylated hydroxy toluene, butylated hydroxyanisole and ethylenediamine tetraacetic acid may be added at levels safe for ingestion to improve storage stability.

According to the present invention, a liquid composition may also contain a buffer such as gluconic acid, lactic acid, citric acid or acetic acid, sodium gluconate, sodium lactate, sodium citrate or sodium acetate. Selection of excipients and the amounts used may be readily determined by the formulation scientist based upon experience and consideration of standard procedures and reference works in the field.

The solid compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable administration in any given case will depend on the nature and severity of the condition being treated, the most preferred route of the present invention is oral. The dosages may be conveniently presented in unit dosage form and prepared by any of the methods well-known in the pharmaceutical arts.

Dosage forms include solid dosage forms like tablets, powders, capsules, suppositories, sachets, troches and lozenges, as well as liquid syrups, suspensions and elixirs.

The dosage form of the present invention may be a capsule containing the composition, preferably a powdered or granulated solid composition of the invention, within either a hard or soft shell. The shell may be made from gelatin and optionally contain a plasticizer such as glycerin and sorbitol, and an opacifying agent or colorant.

The active ingredient and excipients may be formulated into compositions and dosage forms according to methods known in the art.

A composition for tableting or capsule filling may be prepared by wet granulation. In wet granulation, some or all of the active ingredients and excipients in powder form are blended and then further mixed in the presence of a liquid, typically water, that causes the powders to clump into granules. The granulate is screened and/or milled, dried and then screened and/or milled to the desired particle size. The granulate may then be tableted, or other excipients may be added prior to tableting, such as a glidant and/or a lubricant.

A tableting composition may be prepared conventionally by dry blending. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention may include any of the aforementioned blends and granulates that were described with reference to tableting, however, they are not subjected to a final tableting step.

The solid compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable route in any given case will depend on the nature and severity of the condition being treated, the most preferred route of the present invention is oral. The dosages can be conveniently presented in unit dosage form and prepared by any of the methods well-known in the pharmaceutical arts.

Methods of administration of a pharmaceutical composition encompassed by the invention are not specifically restricted, and administered can be in various preparations depending on the age, sex, and symptoms of the patient. For example, tablets, pills, solutions, suspensions, emulsions, granules and capsules may be orally administered. Injection preparations may be administered individually or mixed with injection transfusions such as glucose solutions and amino acid solutions intravenously. If necessary, the injection preparations are administered singly intramuscularly, intracutaneously, subcutaneously or intraperitoneally. Suppositories may be administered into the rectum.

The amount of 1,24(S)-dihydroxy vitamin D₂ contained in a pharmaceutical composition according to the present invention is not specifically restricted, however, the dose should be sufficient to treat, ameliorate, or reduce the targeted symptoms. The dosage of a pharmaceutical composition according to the present invention will depend on the method of use, the age, sex, and condition of the patient.

Having described the invention, the invention is further illustrated by the following non-limiting examples.

### EXAMPLES

In the following examples, “TGA:” designates the weight loss recorded in thermogravimetric analysis, expressed as a percent, over the temperature range of 25 to 200 degrees Celsius measured at a heating rate of 10 degrees per minute using a nominal; sample size of 7-14 mg. Solvent refers to the amount of solvent, express as percent-by-weight in a sample as determined by gas chromatography.

Water content was determined by Karl Fischer titration and is expressed as a percentage by weight.

The crystal forms were identified using an Applied Research Laboratory (SCINTAG) powder X-ray diffractometer model X'TRA equipped with a solid state detector. The crystal samples were analyzed using a round aluminum sample holder with zero background and copper radiation of 1.5418 Å.

### Example 1

### Crystallization from Acetone (Form A)

1,24(S)-dihydroxy vitamin D₂ (6 g) was dissolved in acetone (250 mL), and then the solution was filtered through glass wool to remove undissolved particles. The solution was concentrated under reduced pressure at 30° C. to 163 g then cooled to −18° C. with stirring with a mechanical stirrer and maintained at this temperature 16 hours. The precipitated crystals were recovered by filtration, washed with cold acetone (−18° C., 24 mL), and then dried overnight at 28° C. to obtain Form A (4.5 g, TGA: 5.8%, GC acetone content: 4.6%, Karl Fisher: 1.5%).

### Example 2

### Crystallization from Acetone (Form A)

1,24(S)-dihydroxy vitamin D₂ (1 g) was dissolved in acetone (40 mL). The solution was concentrated under reduced pressure at 30° C. to 34 mL then cooled to 0° C. while stirring with a magnetic stirrer for 1 hour. The solution was cooled to −18° C. and maintained at this temperature for 4 hours. The crystals were recovered by filtration, washed with cold acetone (−18° C.), and then dried overnight at 28° C. to obtain Form A (0.58 g, TGA: 6.1%, GC acetone content: 5.6%).

### Example 3

### Crystallization of Form B

In a 1 L round bottom amber flask, dissolved 1,24(S)-dihydroxy vitamin D₂ (5.8 g), in a solution of 2% water in methyl formate (500 mL methyl formate and 10 ml water), with stirring at 28-30° C. for 30 minutes. The solution was filtered through glass wool to another 1 L round bottom amber flask, and the flask was washed with methyl formate (40 mL), which was also filtered through the glass wool.

The solution was then cooled to 0° C. with stirring under nitrogen for 1 hour and then cooled to −18° C. for 1 hour. The solution was then stirred at −18° C. under nitrogen for 16-19 hours.

The mixture was filtered on a Buchner funnel and washed with cold (below −15° C., 2×20 mL) methyl formate. The solid was transferred to a round dish (d=7-8 cm) and dried in vacuum oven under high vacuum (less than 5 mm Hg) at 28° C. for 6-8 hours.

### Example 4

### Crystallization from Acetone/Water (Form B)

1,24(S)-dihydroxy vitamin D₂ (2 g) was dissolved in acetone (100 mL), and then water (35 mL) was added. The clear solution was filtered through glass wool to remove undissolved particles. The solution was stirred and cooled to 0° C. for 1.5 hours. The crystals were recovered by filtration, washed with cold acetone/water solution (0° C., 10 mL), and then dried overnight at 28° C. to obtain Form B (1.4 g, TGA: 6.2%, Karl Fisher: 3.7%).

### Example 5

### Crystallization from Ethyl Acetate (Form C)

1,24(S)-dihydroxy vitamin D₂ (3.3 g) was dissolved in ethyl acetate (110 mL). The clear solution was concentrated under reduced pressure at 30° C. to 70 g then cooled to −18° C. while stirring with a mechanical stirrer for 18 hours. The crystals were recovered by filtration, washed with cold ethyl acetate (−18° C., 16 mL), and then dried overnight at 28° C. to obtain Form C (2 g, TGA: 1.3%, GC ethyl acetate content: 0.4%).

### Example 6

### Crystallization from Methyl Formate (Form D)

1,24(S)-dihydroxy vitamin D₂ (1 g) was dissolved in methyl formate (85 mL). The solvent was concentrated under reduced pressure at 30° C. to 68 ml then cooled to 0° C. while stirring with a magnetic stirrer for 1 hour. Then, the solution was cooled to −18° C. overnight. The crystals were recovered by filtration, washed with cold methyl formate (−18° C.), and then dried for 6 hours at 28° C. to obtain Form D (0.62 g, TGA: 2.9%, GC methyl formate content: 140 ppm).

### Example 7

### Crystallization from Ethyl Acetate (Form D)

1,24(S)-dihydroxy vitamin D₂ (1.06 g) was dissolved ethyl acetate (30 mL). The solvent was concentrated under reduced pressure at 30° C. to 20.3 ml, and then cooled to 0° C. while stirring with a magnetic stirrer for 1 hour. Then, the solution was cooled to −18° C. for 24 hours. The crystals were recovered by filtration, washed with cold ethyl acetate (−18° C.), and then dried overnight at 28° C. to obtain Form D (0.6 g, TGA: 1.5%, GC ethyl acetate content: 0.3%).

TABLE 1
Characterization of Crystalline Forms
Peaks are measured in degrees two-theta ± 0.2 degrees two-theta (2θ)
Peaks in bold are the most characteristic peaks.

| | Form | | | |
|---|---|---|---|---|
| | A | B | C | D |
| Characteristic | 7.2 | 8.0 | 6.2 | 6.0 |
| XRD peaks | 12.0 | 10.1 | 13.4 | 13.4 |
| | 14.2 | 13.6 | 14.7 | 14.5 |
| | 14.8 | 15.3 | 15.6 | 15.0 |
| | 16.2 | 15.6 | 16.2 | 15.6 |
| | 16.6 | 16.2 | 17.1 | 16.4 |
| | 18.4 | 17.1 | 18.4 | 16.8 |
| | 22.1 | 17.6 | 18.8 | 17.8 |
| | 23.0 | 20.4 | | 20.5 |
| | 23.8 | 22.1 | | 21.8 |
| | 24.7 | 23.9 | | 23.1 |
| | 27.9 | 30.8 | | 24.6 |
| | | | | 24.9 |
| Water content | — | 2%-4% | 1%-2% | 2%-3% |
| (Karl Fisher) | | | | |
| Solvent content | 6% (acetone) | — | — | — |
| (TGA/GC) | | | | |
| Definition of | hemi-acetonate | monohydrate | hemihydrate | sesqui- |
| solvated state | | | | hydrate |

TABLE 2
Stability of the forms
Forms exposed to 100% relative humidity for 1 week.

| | Initial form | | |
|---|---|---|---|
| | A | B | C |
| Resulting Form | B | B | C |
| after exposure to 100% relative | | | |
| humidity | | | |

Having thus described the invention with reference to particular preferred embodiments and illustrative non-limiting examples, those in the art will appreciate modifications to the invention as described and illustrated that do not depart from the scope of the invention as disclosed in the specification.

What is claimed is:

1. 1,24(S)-dihydroxy vitamin D₂ hydrate.

2. The 1,24(S)-dihydroxy vitamin D₂ hydrate of claim 1, containing between about 1% to about 4% water.

3. The 1,24(S)-dihydroxy vitamin D₂ hydrate of claim 1, wherein the hydrate is selected from the group consisting of: a monohydrate, a hemihydrate and a sesquihydrate.

4. 1,24(S)-dihydroxy vitamin D₂ solvate.

5. The 1,24(S)-dihydroxy vitamin D₂ solvate of claim 4, wherein the solvate is an acetonate.

6. The 1,24(S)-dihydroxy vitamin D₂ solvate of claim 5, wherein the solvate is a hemi-acetonate.

7. A crystalline form of 1,24(S)-dihydroxy vitamin D₂ characterized by X-ray reflections at about 14.2, 16.2, 16.6, 18.4, and 22.1°±0.2° 2θ.

8. The crystalline form of 1,24(S)-dihydroxy vitamin D₂ of claim 7, further characterized by X-ray reflections at about 7.2, 12.0, 14.8, 23.0, 23.8, 24.7, and 27.9°±0.2° 2θ.

9. The crystalline form of 1,24(S)-dihydroxy vitamin D₂ of claim 8, having a powder X-ray diffraction diagram substantially as shown in FIG. 1.

10. The crystalline form of 1,24(S)-dihydroxy vitamin D₂ of claim 7, wherein the crystalline form is a hemi-acetonate.

11. A method of making the crystalline form of 1,24(S)-dihydroxy vitamin D₂ of claim 7 comprising the steps of: providing a solution of 1,24(S) dihydroxy vitamin D₂ in acetone, cooling the solution to a temperature of about 0° C. to about −20° C., maintaining the resulting cooled mixture for at least about 15 hours to obtain a precipitate, and recovering the crystalline form.

12. The method of claim 11, wherein the solution is cooled to a temperature of about −18° C.

13. The method of claim 11, wherein the solution is initially cooled to a temperature of about 0° C. and maintained for a period of about 1 hour, followed by further cooling to a temperature of about −18° C.

14. The method of claim 11, wherein prior to cooling, the solution is concentrated to about 70% to about 85% of its initial volume.

15. A crystalline form of 1,24(S)-dihydroxy vitamin D₂ characterized by X-ray reflections at about 13.6, 15.3, 16.2, 17.1, and 17.6°±0.2° 2θ.

16. The crystalline form 1,24(S)-dihydroxy vitamin D₂ of claim 15, further characterized by X-ray reflections at about 13.6, 15.3, 16.2, 17.1, and 17.6°±0.2° 2θ.

17. The crystalline form of 1,24(S) dihydroxy vitamin D₂ of claim 16 having a powder X-ray diffraction diagram substantially as shown in FIG. 2.

18. The 1,24(S)-dihydroxy vitamin D₂ solvate of claim 15, wherein the crystalline form is a monohydrate.

19. A method of making the crystalline form of 1,24(S)-dihydroxy vitamin D₂ of claim 15 comprising the steps of: providing a solution of 1,24(S)-dihydroxy vitamin D₂ in a mixture of water and methyl formate, cooling the provided solution to a temperature of about 0° C. to about −20° C., maintaining the reaction mixture for a period of about 16 to about 19 hours to obtain a precipitate, and recovering the crystalline form.

20. The method of claim 19, wherein the solution is initially cooled to a temperature of about 0° C. and maintained for a period of about 1 hour, followed by further cooling to a temperature of about −18° C.

21. The method of claim 19, wherein the water and methyl formate are about 1:50 on a volume basis.

22. A method of making the crystalline form of 1,24(S)-dihydroxy vitamin D₂ of claim 15 comprising the steps of: providing a solution of 1,24(S)-dihydroxy vitamin D₂ in a mixture of acetone and water, cooling the provided solution to a temperature of about 0° C. for a period of about 1.5 hours to obtain a precipitate, and recovering the crystalline form.

23. The method of claim 22, wherein the water and acetone are about 1:3 on a volume basis.

24. A crystalline form of 1,24(S)-dihydroxy vitamin D₂ characterized by X-ray reflections at about 14.7, 15.6, 16.2, and 17.1°±0.2° 2θ.

25. The crystalline form of 1,24(S)-dihydroxy vitamin D₂ of claim 24, further characterized by X-ray reflections at about 6.2, 13.4, 18.4, and 18.8°±0.2° 2θ.

26. The crystalline form of 1,24(S)-dihydroxy vitamin D₂ of claim 25, having a powder x-ray diffraction diagram substantially as shown in FIG. 3.

27. The crystalline form of 1,24(S)-dihydroxy vitamin D₂ of claim 24, wherein the crystalline form is a hemihydrate.

28. A method of making the crystalline form of 1,24(S)-dihydroxy vitamin D₂ of claim 24 comprising the steps of: providing a solution of 1,24(S) dihydroxy vitamin D₂ in ethyl acetate, cooling the solution to a temperature of about −10° C. to about −20° C., maintaining the cooled solution for about 5 to about 20 hours to obtain a precipitate, and recovering the crystalline form.

29. The method of claim 28, wherein the solution is cooled to a temperature of about −18° C.

30. The method of claim 28, wherein the cooled solution is maintained for about 18 hours.

31. The method of claim 28, wherein prior to cooling, the solution is concentrated to about 60% to about 80% of its initial volume.

32. A crystalline form of 1,24(S)-dihydroxy vitamin D₂, characterized by X-ray reflections at about 13.4, 14.5, 15.0, and 16.8°±0.2° 2θ.

33. The crystalline form of 1,24(S)-dihydroxy vitamin D₂ of claim 32, further characterized by X-ray reflections at about 6.0, 15.6, 16.4, 17.8, 20.5, 21.8, 23.1, 24.6, and 24.9°±0.2° 2θ.

34. The crystalline form of 1,24(S)-dihydroxy vitamin D₂ of claim 33 having a powder X-ray diffraction diagram substantially as shown in FIG. 4.

35. The crystalline form of 1,24(S)-dihydroxy vitamin D₂ of claim 32, wherein the crystalline form is a sesquihydrate.

36. A method of making the crystalline form of 1,24(S)-dihydroxy vitamin D₂ of claim 32 comprising the steps of: providing a solution of 1,24(S)-dihydroxy vitamin D₂ in a solvent selected from methyl formate or ethyl acetate, cooling the provided solution to a temperature of about 0° C. for about 1 hour, further cooling to a temperature of about −10° to about −20° C., maintaining the reaction mixture for about 16 to about 19 hours to obtain a precipitate, and recovering the crystalline form.

37. The method of claim 28, wherein prior to cooling, the solution is concentrated to about 60% to about 80% of its initial volume.

38. A pharmaceutical or nutraceutical composition prepared by combining at least one pharmaceutically acceptable excipient with at least one of the crystalline forms of 1,24(S)-dihydroxy vitamin D₂ of any one of claims 1, 4, 7, 15, 24 and 32.
